(19) United States
(12) Patent Application Publication
(10) Pub. No.: US 2024/0226210 A9
(43) Pub. Date: Jul. 11, 2024

(54) ONCOLYTIC VIRUS COMBINED WITH CAR T CELLS FOR ANTI-TUMOR THERAPY

(71) Applicants: Broaden Bioscience and Technology Corp, KATY, TX (US); Orgenesis Inc, GERMANTOWN, MD (US); Joint BioScinces, Shanghai (CN)

(72) Inventors: BUO CHEN, KATY, TX (US); GUOQING ZHOU, SHANGHAI (CN); XIANGGUM LI, BEIJING (CN)

(73) Assignees: Broaden Bioscience and Technology Corp, Katy, TX (US); Orgenesis, Inc, Germantown, MD (US); Joint BioSciences Ltd, Shanghai (CN)

(21) Appl. No.: 18/493,787

(22) Filed: Oct. 24, 2023

Related U.S. Application Data
(60) Provisional application No. 63/380,631, filed on Oct. 24, 2022.

(51) Int. Cl. A61K 35/768; A61K 9/00; A61K 35/17; A61K 39/00; A61K 45/06; A61P 35/00; C07K 14/145; C07K 14/54; C07K 16/28

(52) U.S. Cl. CPC A61K 35/768; A61K 9/0019; A61K 35/17; A61K 39/4611; A61K 39/4631; A61K 39/464412; A61K 45/06; A61P 35/00; C07K 14/145; C07K 14/5434; C07K 16/2803; A61K 2239/13; A61K 2239/48

(57) ABSTRACT

The present disclosure provides methods of treating cancer using complementary transgenic oncolytic viruses and genetically engineered CAR T cells. In one embodiment, the oncolytic virus comprises nucleotide sequences encoding CD19, or CD19 and IL-12, and the genetically engineered T cells express a chimeric antigen receptor that recognizes CD19.

### SEQUENCE LISTING

The instant application contains a Sequence Listing which has been submitted electronically in XML file format and is hereby incorporated by reference in its entirety. Said XML copy, created on Sep. 20, 2022, is named P-608341-USP_SL.xml and is 16,181 bytes in size.

### FIELD OF THE INVENTION

The present disclosure is generally related to the field of anti-tumor therapy. In one embodiment, the present disclosure provides methods of treating cancer using complementary transgenic oncolytic viruses and genetically engineered CAR T cells.

### BACKGROUND OF THE INVENTION

Acute lymphoblastic leukemia (ALL) is a malignant clonal disease of hematopoietic stem cells. At present, B cell malignancies (including acute B-lymphocytic leukemia (B-ALL), chronic non-lymphocytic leukemia (B-CLL), non-Hodgkin's lymphoma, etc.) are currently the best types of diseases for which chimeric antigen receptor (CAR) T cell technology is applied. In recent years, chimeric antigen receptor modified T cells (anti-CD19 or anti-CD 22 CAR-T cells) targeting CD19 or CD22 have shown good efficacy in the treatment of acute B lymphocyte malignancies. It has been shown that the 3-month complete remission rate for acute B-ALL patients treated with CD19 CAR-T cells is above 80-90%. Although the therapeutic effect for B cell lymphoma is not as good as that of B-ALL, the complete remission rate can reach about 54%. In view of the excellent performance of anti-CD19 CAR-T in the treatment of B-cell malignant tumors, the United States has so far approved five CAR-T products targeting CD19. CAR T therapies targeting other antigens such as CD33 or CD123 are also under intense development.

Although CAR-T therapy has achieved a high rate of complete remission in the treatment of B-cell malignancies, clinical data show that a considerable proportion of patients who have achieved complete remission after CAR-T cell therapy relapse within 9-12 months. For example, for B-ALL patients treated with anti-CD19 CAR-T treatment, the 12-month recurrence rate is as high as 43-55%, and 20-30% of these recurrence cases has the CD19 antigen lost. Due to the loss of the target antigen, the patient loses the option of retreatment with anti-CD19 CAR-T. At present, there is no effective treatment for these patients who have relapsed, and the mortality rate is very high. This is currently an important unmet need in clinical practice.

Although CAR-T therapy has shown promising results in treating B-cell malignancies, it is still lacking effective CAR-T therapy for acute myeloid leukemia (AML). The main reason is that AML does have specific target protein such as CD19 for B-ALL. Presently, the CAR-T products designed for AML mainly target at Lewis-Y, CLL1, CD33, CD123, CD44-V6 or FLT-3. Since hematopoietic stem cells also express CD33 and CD123, toxicity in the hematopoietic system is frequently observed after infusion of the CD33 or CD123 CAR-T cells. The effectiveness of other targets are still yet to be confirmed. Consequently, there is still an unmet need for CAR-T therapy for AML.

Oncolytic viruses are naturally occurring or genetically modified viruses that can infect tumor cells, replicate in tumor cells, and ultimately kill tumor cells without harming healthy cells. It has been proposed that Vesicular stomatitis virus (VSV) can be used as an oncolytic virus to treat tumors. This virus would not interact with the endogenous IFN-β in normal cells, and can only selectively expand and grow in tumor cells.

VSV can express a variety of cell surface molecules including low-density lipoprotein receptor, phosphatidylserine, sialolipid and heparan sulfate, and can attach to the cell surface through such molecules. Compared with other oncolytic cell virus platforms currently under development, VSV has the following advantages: (i) the genome is small, the replication time is short, and the cross-synaptic speed is fast; (ii) exogenous gene expression is extremely high, so it can have a high titer, allowing large-scale production; (iii) there is an independent cell cycle, and there is no risk of transformation in the cytoplasm of the host cell. This oncolytic virus will not be integrated into the DNA, and after attenuation, nervous system inflammation caused by the wild-type virus is avoided. In view of the above characteristics, VSV has a good potential in tumor immunotherapy.

Although VSV has the above advantages, when VSV is used alone for tumor immunotherapy, there are certain technical bottlenecks. In addition to the issue of cell specificity, because of the existence of the inhibitory microenvironment within the tumor, the virus cannot repeatedly exert its efficacy. Consequently, there is a need to develop new strategy to combine application of recombinant VSV oncolytic virus with other treatments, aiming to exert a synergistic effect and better anti-tumor effect.

### SUMMARY OF THE INVENTION

In one embodiment, the present disclosure provides use of a composition for treating cancer in an individual, the composition comprising (i) an oncolytic virus comprising nucleotide sequences encoding an antigen, or an antigen and IL-12; and (ii) genetically engineered T cells expressing a chimeric antigen receptor (CAR) that recognizes the antigen. In one embodiment, the antigen is CD19. In one embodiment, the antigen is a truncated CD19, which only contains extracellular domain and transmembrane domain. In one embodiment, the CD19 antigen comprises the amino acid sequence of SEQ ID NO:5, 6 or 7. In one embodiment, the anti-CD19 CAR comprises the amino acid sequence of SEQ ID NO:10. In one embodiment, the T cells are obtained from the individual. Representative examples of oncolytic virus include, but are not limited to, vaccinia virus, reovirus, Seneca Valley virus (SVV), vesicular stomatitis virus (VSV), Newcastle disease virus (NDV), herpes simplex virus (HSV), measles virus, adenovirus, or poxvirus. In one embodiment, the oncolytic virus is vesicular stomatitis virus.

In one embodiment, the oncolytic virus comprises M protein having one or more amino acid mutations N32S, N49D, M51R, H54Y, V221F, V225I, and S226R as compared with SEQ ID NO:1, i.e., the amino acid substitution(s) of the M protein comprises one or more of the following: asparagine in position 32 was mutated to serine (N32S); asparagine in position 49 was mutated to aspartic acid (N49D); methionine in position 51 was mutated to arginine (M51R); histidine in position 54 was mutated to tyrosine (H54Y); valine in position 221 was mutated to phenylalanine (V221F); valine at position 225 was mutated to isoleucine (V225I); and serine at position 226 was mutated to arginine (S226R).

In one embodiment, the mutated M protein comprises the amino acid sequence of SEQ ID NO:2.

In one embodiment, the oncolytic virus comprises G protein having one or more amino acid mutations V53I, A141V, D172Y, K217E, D232G, V331A, V371E, G436D, T438S, F453L, T471I, and Y487I; for example, valine (V) at position 53 was mutated to isoleucine (I) (V53I); alanine (A) at position 141 was mutated to valine (V) (A141V); aspartic acid (D) at position 172 was mutated to tyrosine (Y) (D172Y); lysine (K) at position 217 was mutated to glutamic acid (E) (K217E); aspartic acid (D) at position 232 was mutated to glycine (G) (D232G); valine at position 331 (V) was mutated to alanine (A) (V331A); valine (V) at position 371 was mutated to glutamic acid (E) (V371E); glycine (G) at position 436 was mutated to aspartic acid (D) (G436D); threonine (T) at position 438 was mutated to serine (S) (T438S); phenylalanine (F) at position 453 was mutated to leucine (L) (F453L); threonine (T) at position 471 was mutated to isoleucine (I) (T471I); tyrosine (Y) at position 487 was mutated to isoleucine (I) (Y487I).

In one embodiment, the mutated G protein comprises the amino acid sequence of SEQ ID NO:4.

In one embodiment, the oncolytic virus is administered to the individual parenterally or intratumorally. In one embodiment, the engineered T cells are administered to the individual via intravenous, intra-arterial, or intralymphatic delivery.

In one embodiment, the individual is further treated with other treatments such as, but not limited to, surgery, immunotherapy, chemotherapy, radiation therapy, targeted therapy, hormone therapy, stem cell therapies, or blood transfusions.

In one embodiment, the individual is having a cancer such as acute lymphoblastic leukemia, acute B-lymphocytic leukemia, chronic non-lymphocytic leukemia, non-Hodgkin's lymphoma, multiple myeloma, breast cancer, or solid cancers such as colon cancer, liver cancer, pancreatic cancer.

In another embodiment, the present disclosure provides a method of treatment of an individual having a cancer, comprising the steps of: (i) administering to the individual an oncolytic virus comprising nucleotide sequences encoding an antigen, or an antigen and IL-12; and (ii) administering to the individual genetically engineered T cells expressing a chimeric antigen receptor that recognizes the antigen. In one embodiment, the antigen is CD19. In one embodiment, the CD19 antigen comprises the amino acid sequence of SEQ ID NO:5, or 6, or 7. In one embodiment, the anti-CD19 CAR comprises the amino acid sequence of SEQ ID NO:10. In one embodiment, the T cells are obtained from the individual. Representative examples of oncolytic virus include, but are not limited to, vaccinia virus, reovirus, Seneca Valley virus (SVV), vesicular stomatitis virus (VSV), Newcastle disease virus (NDV), herpes simplex virus (HSV), measles virus, adenovirus, or poxvirus. In one embodiment, the oncolytic virus is vesicular stomatitis virus.

In one embodiment, the oncolytic virus comprises M protein having one or more amino acid mutations N32S, N49D, M51R, H54Y, V221F, V225I, and S226R as compared with SEQ ID NO:1, i.e., the amino acid substitution(s) of the M protein comprises one or more of the following: asparagine in position 32 was mutated to serine (N32S); asparagine in position 49 was mutated to aspartic acid (N49D); methionine in position 51 was mutated to arginine (M51R); histidine in position 54 was mutated to tyrosine (H54Y); valine in position 221 was mutated to phenylalanine (V221F); valine at position 225 was mutated to isoleucine (V225I); and serine at position 226 was mutated to arginine (S226R).

In one embodiment, the mutated M protein comprises the amino acid sequence of SEQ ID NO:2.

In one embodiment, the oncolytic virus comprises G protein having one or more amino acid mutations V53I, A141V, D172Y, K217E, D232G, V331A, V371E, G436D, T438S, F453L, T471I, and Y487I as described above. In one embodiment, the mutated G protein comprises the amino acid sequence of SEQ ID NO:4.

In one embodiment, the oncolytic virus is administered to the individual parenterally or intratumorally. In one embodiment, the engineered T cells are administered to the individual via intravenous, intra-arterial, or intralymphatic delivery.

In one embodiment, the individual is further treated with other treatments such as, but not limited to, surgery, immunotherapy, chemotherapy, radiation therapy, targeted therapy, hormone therapy, stem cell therapies, or blood transfusions.

In one embodiment, the individual is having a cancer such as acute lymphoblastic leukemia, acute B-lymphocytic leukemia, chronic non-lymphocytic leukemia, non-Hodgkin's lymphoma, multiple myeloma, breast cancer, or solid cancers such as colon cancer, liver cancer, pancreatic cancer.

These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 shows a map of the VSV vector carrying full length CD19 or truncated CD19 polypeptide.

FIG. 2 shows a map of the VSV vector carrying IL12 and full length CD19 or truncated CD19 polypeptide

FIG. 3 shows a map of one embodiment of an anti-CD19 CAR.

FIG. 4 shows cell viability of K562 cells upon incubation at different MOIs with recombinant VSV virus expressing CD19.

FIG. 5 shows CD19 expression on K562 cells incubated at different MOIs with recombinant VSV virus expressing CD19.

FIG. 6 shows in vitro killing of CD19 CART cells against target cells expressing full-length (CD19-VSV) or truncated CD19 (TrunCD19-VSV). The cell groups are: CD19 CAR-T cells only, CD19 CAR-T cells and target cells loaded with CD19 full-length recombinant VSV (CD19 CAR-T cells+CD19-VSV), CD19 CAR-T cells and target cells loaded with recombinant VSV expressing truncated CD19 (CD19 CAR-T cells+TrunCD19-VSV), Recombinant VSV virus only (CD19-VSV), recombinant VSV virus with truncated CD19 only (TrunCD19-VSV), and un-transduced T cells (UTD). E:T ratios: 1.25:1, 2.5:1, and 5:1.

FIG. 7 shows CD19 expression in CD19 knockout mouse B-cell lymphoma cell line Raji (left panel) or wild-type Raji cells (right panel).

FIG. 8 shows tumor volume in mice treated with: CD19 CAR-T cells only, CD19 CAR-T cells and VSV expressing full-length CD19 (CD19 CAR T+CD19-VSV), CD19 CAR-T cells and VSV expressing truncated CD19 (CD19 CAR T+TrunCD19-VSV), VSV expressing full-length CD19 (CD19-VSV), VSV expressing truncated CD19 (TrunCD19-VSV), or un-transduced T cells (UTD).

FIG. 9 shows induction of CD19 expression on Kasumi-1 cells, K562 cells, or THP1 cells by recombinant VSV virus encoding CD19.

### DETAILED DESCRIPTION OF THE INVENTION

Various embodiments are directed towards methods of treatments for cancer utilizing complementary oncolytic virus and CAR T cells. In various embodiments, the oncolytic viruses are engineered to comprise nucleotide sequences encoding an antigen, or an antigen and IL-12. In one embodiment, the antigen is CD19.

In several embodiments, an oncolytic virus is engineered to express an antigen, or an antigen and IL-12, and induce transgenic expression of an ectopic antigen on the surface of a neoplastic and/or a cancer cell. In several embodiments, T cells are engineered to express a CAR that is capable of recognizing the ectopically expressed antigen. In several embodiments, an individual with a cancer or neoplasm is treated by administering to the individual an oncolytic virus to induce (i) (ectopic) antigen expression on the outer surface of the cancer cell or tumor cell membrane or within the tumor cell; or (ii) tumor cell expression of IL-2 and (ectopic) antigen expression. Complimentary CAR T cells that recognize the (ectopic) antigen expressed by the oncolytic virus are then administered to the individual. Those CAR T cells can induce an immune response against the cancer or neoplastic cells.

In one embodiment, the compositions and methods provided herein can be used to treat relapsed cancer cases in which the target antigen or tumor antigen is lost or has low expression. For example, the compositions and methods provided herein can be used to treat relapsed cancer cases in which the CD19 antigen is lost or expression of CD19 is low, comprising a combined use of engineered oncolytic viruses and engineered CAR T cells. In one embodiment, the oncolytic viruses are engineered to express full length or truncated CD19 in the tumor cells. In one embodiment, the truncated CD19 only contains extracellular domain and transmembrane domain of CD19. Thus, when the engineered oncolytic viruses infect malignant tumor cells, on one hand the malignant cells are lysed, while on the other hand the engineered oncolytic viruses would restore high expression of CD19 on the tumor cells. Moreover, the cancer patient would be treated by re-infusion of CD19 CAR T cells. In order to increase the therapeutic effect, the present invention also adds the cytokine IL-12 to the oncolytic virus vector. IL-12 is a pleiotropic cytokine with anti-tumor effect; its anti-tumor mechanism includes the following aspects: (a) promote the production of IFN-γ; (b) increase the activation of NK cells and CD4 T cells; (c) increase the cytotoxicity of CD8 T cells; (d) promote anti-angiogenic effects by inducing the production of anti-angiogenic cytokines and chemokines; (e) participate in the remodeling of tumor extracellular matrix and so on. Thus, the present invention combines three anti-tumor mechanisms and the anti-tumor effects would be synergistic. The present invention not only allows CD19 antigen-negative relapsed patient to have the opportunity of being treated again, it will also exert a stronger anti-tumor effect, so that the patient will obtain benefit to a greater extent.

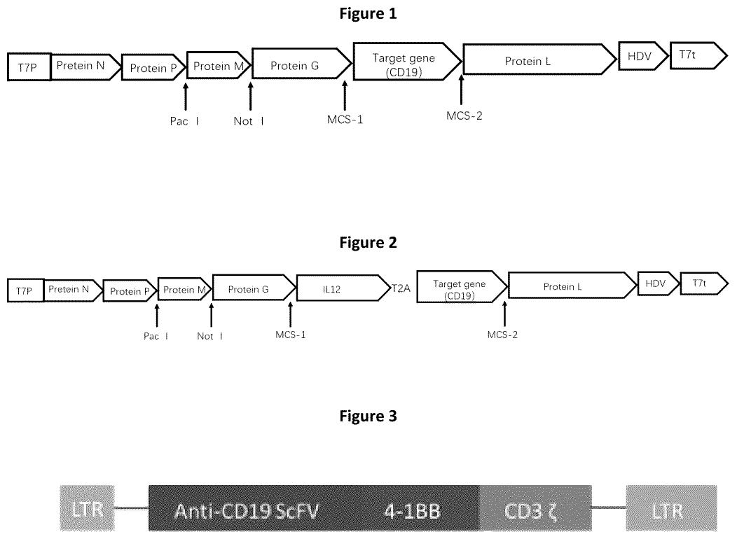

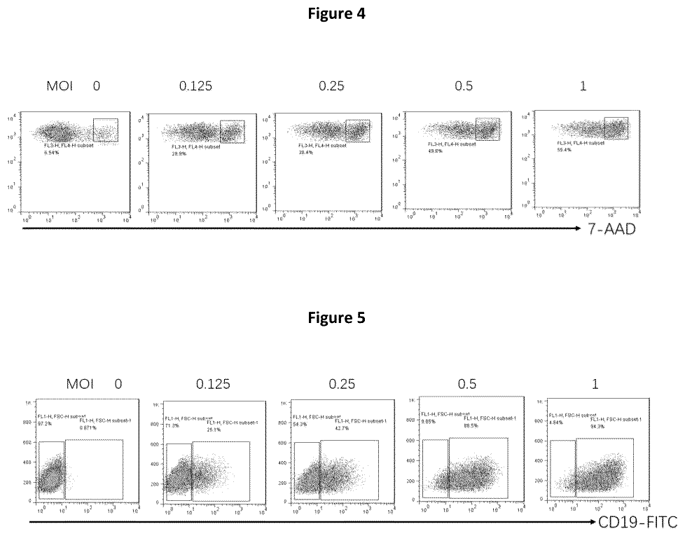

Thus, in one embodiment to solve the problem of loss of target antigen or unclear target antigen in CD19 CAR T treatment of B cell malignant tumors, the present invention provides a combined application of (i) recombinant oncolytic virus (e.g. vesicular stomatitis virus, VSV) engineered to express a full length or truncated CD19, or engineered to express IL-12 and a full length or truncated CD19, and (ii) CD19 CAR T cells. The technical solutions are as follows:
1. In order to better ensure safety, the recombinant oncolytic virus (e.g. VSV) used in the present invention is an attenuated strain obtained by point mutation of its M protein. In one embodiment, amino acid substitution of M protein comprises one or more of N32S, N49D, M51R, H54Y, V221F, V225I, and S226R.
2. A virus strain with G protein mutations was constructed on the basis of the above attenuated strain, and the G protein mutation sites comprise one or more of the following: valine (V) at position 53 was mutated to isoleucine (I) (V53I); alanine (A) at position 141 was mutated to valine (V) (A141V); aspartic acid (D) at position 172 was mutated to tyrosine (Y) (D172Y); lysine (K) at position 217 was mutated to glutamic acid (E) (K217E); aspartic acid (D) at position 232 was mutated to glycine (G) (D232G); valine at position 331 (V) was mutated to alanine (A) (V331A); valine (V) at position 371 was mutated to glutamic acid (E) (V371E); glycine (G) at position 436 was mutated to aspartic acid (D) (G436D); threonine (T) at position 438 was mutated to serine (S) (T438S); phenylalanine (F) at position 453 was mutated to leucine (L) (F453L); threonine (T) at position 471 was mutated to isoleucine (I) (T471I); tyrosine (Y) at position 487 was mutated to isoleucine (I) (Y487I).
3. On the basis of the attenuated oncolytic virus strain described herein, a recombinant VSV containing sequences encoding IL-12 and full length or truncated CD19 was constructed. IL-12 can promote the proliferation and differentiation of T cells and is widely used for cancer treatment, such as melanoma or kidney cancer. VSV contains 5 functional proteins. In the present invention, sequences encoding IL12 and CD19 were inserted between the sequences encoding the G and L proteins. The maps of the engineered VSV are shown in FIG. 1 and FIG. 2.
4. CD19 CAR lentiviral vector construction and CD19 CAR T cell preparation. In one embodiment, the CD19 CAR comprises a CD8 signal peptide, CD19 antibody (FMC63) single chain variable region, hinge region, transmembrane region, 4-1BB intracellular region and CD3ζ. The sequences are connected in series and constructed in an expression lentiviral vector with suitable restriction sites, that is, the CD19 CAR lentiviral vector. A map of the CD19 CAR is shown in FIG. 3.
5. In one embodiment, the prepared recombinant VSV oncolytic viruses carrying full length or truncated CD19 are applied simultaneously with CD19 CAR T cells. In another embodiment, the prepared recombinant VSV oncolytic viruses carrying full length or truncated CD19 are applied sequentially with CD19 CAR T cells. For example, the oncolytic viruses are administered before or after or at the same time of the administration of the CD19 CART cells.

In one embodiment, the method disclosed herein comprises: administering the recombinant VSV oncolytic viruses described herein through intravenous injection or one or more intratumoral injections, together with intravenous infusion of CD19 CAR-T cells at the same time. In another embodiment, the CD19 CAR-T cells are administered by intravenous infusion 12-72 hours after the recombinant VSV oncolytic viruses are administered, or vice versa. For example, the CD19 CAR-T cells are administered first by intravenous infusion, then the recombinant VSV oncolytic viruses described herein are administered through intravenous injection or one or more intratumoral injections. In another embodiment, the recombinant VSV oncolytic virus described herein and the CD19 CAR-T cells are first incubated or cultured in vitro, then the CD19 CAR-T cells are used to carry the oncolytic virus to the tumor site for treatment.

The term oncolytic virus is utilized to describe viruses or viral vectors that preferentially infect or transduce neoplastic or cancer cells. In some instances, infection or transduction of a neoplastic cells with oncolytic virus can result in the lysis and/or death the cell, but lysis and/or death is not necessarily required. In various embodiments described herein, an oncolytic virus is engineered to induce ectopic expression of a CAR T cell target antigen. Any appropriate oncolytic virus can be utilized to deliver transgene expression to neoplastic cells. In some embodiments, an oncolytic virus is engineered through generally known techniques to achieve desired attributes, which may include cellular tropism, virus attenuation, and enhanced transgene expression. A number of modified viruses or viral vectors can be utilized as oncolytic viruses, including (but not limited to) reovirus, Seneca Valley virus (SVV), vesicular stomatitis virus (VSV), Newcastle disease virus (NDV), herpes simplex virus (HSV), measles virus, adenovirus, and poxvirus (e.g., vaccinia virus).

In one embodiment, the oncolytic virus is engineered to carry sequences encoding a target antigen to be recognized by the CAR T cells. In some embodiments, the target antigen is exogenous, meaning the antigen is from a different species. In some embodiments, the target antigen is an endogenous antigen, such as one typically expressed on a tumor cell. In one embodiment, the oncolytic virus is engineered to carry sequences encoding a target antigen such as CD19. In some embodiments, the target antigens are tumor associated antigens (TAAs). In some embodiments, the TAAs encompass molecules, or a portion thereof, that are displayed on the surface of a cell or present within the milieu of a tumor, e.g. within the tumor micro-environment. In some embodiments, the cell is a tumor cell.

In some embodiments, a TAA encompasses a cell surface tumor associated antigen. In some embodiments, the cell is a non-tumor cell present in the milieu of a tumor, for example but not limited to, a cell present within vasculature tissue associated with a tumor or cancer. In some embodiments, a TAA is an angiogenic antigen in a tumor micro-environment. In some embodiments, a TAA is an antigen on a blood vessel in a tumor micro-environment. In some embodiments, the cell is a stromal cells present in the milieu of a tumor. In some embodiments, a TAA is a stromal cell antigen within a tumor micro-environment. In some embodiments, a TAA encompasses an extracellular epitope of a tumor cell-surface antigen. In some embodiments, a TAA encompasses an extracellular matrix antigen.

In some embodiments, a TAA comprises an antigen present in a tumor micro-environment (TME). In some embodiments, a TAA comprises a molecule secreted by a tumor cell into the TME. In some embodiments, a TAA comprises an effector molecule secreted by a tumor cell into the TME. In some embodiments, a TAA comprises an effector molecule secreted by a tumor cell into the TME in order to downregulate or inhibit the activity of cytotoxic natural killer (NK) or T cells. In some embodiments, a TAA comprises soluble activating receptor ligand secreted by a tumor cell into the TME in order to block the recognition of the tumor cell by a NK cell or T cell.

In some embodiments, examples of TAAs include, but are not limited to, 5T4, ROR1, EGFR, FcγRI, FcγRIIa FcγRIIb FcγRIIIa FcγRIIIb, CD28, CD137, CTLA-4, FAS, FAP (Fibroblast activation protein), LGR5, C5aR1, A2AR, fibroblast growth factor receptor 1 (FGFR1), FGFR2, FGFR3, FGFR4, glucocorticoid-induced TNFR-related (GITR) protein, lymphotoxin-beta receptor (LTβR), toll-like receptors (TLR), tumor necrosis factor-related apoptosis-inducing ligand-receptor 1 (TRAIL receptor 1), TRAIL receptor 2, prostate-specific membrane antigen (PSMA) protein, prostate stem cell antigen (PSCA) protein, tumor-associated protein carbonic anhydrase IX (CAIX), epidermal growth factor receptor 1 (EGFR1), EGFRvIII, human epidermal growth factor receptor 2 (Her2/neu; Erb2), ErbB3 (HER3), Folate receptor, ephrin receptors, PDGFRa, ErbB-2, CD2, CD20, CD22, CD30, CD33, CD40, CD37, CD38, CD70, CD74, CD56, CD80, CD86, CD123, CCAM5, CCAM6, BCMA, p53, cMet (tyrosine-protein kinase Met) (hepatocyte growth factor receptor) (HGFR), MAGE-A1, MAGE-A2, MAGE-A3, MAGE-A4, MAGE-A6, MAGE-A10, MAGE-A12, BAGE, DAM-6, DAM-10, GAGE-1, GAGE-2, GAGE-8, GAGE-3, GAGE-4, GAGE-5, GAGE-6, GAGE-7B, NA88-A, NY-ESO-1, BRCA1, BRCA2, MART-1, MC1R, Gp100, PSA, PSM, Tyrosinase, Wilms' tumor antigen (WT1), TRP-1, TRP-2, ART-4, CAMEL, Cyp-B, hTERT, hTRT, iCE, MUC1, MUC2, P-cadherin, Myostatin (GDF8), Cripto (TDGF1), MUC5AC, PRAME, P15, RU1, RU2, SART-1, SART-3, WT1, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pm1/RARα, TEL/AML1, CD28, CD137, CanAg, Mesothelin, DR5, PD-1, PD1L, IGF-1R, CXCR4, Neuropilin 1, Glypicans, EphA2, CD138, B7-H3, B7-H4, gpA33, GPC3, SSTR2, or VEGF-R2.

In several embodiments, an individual's own T cells are engineered to express a CAR capable of recognizing a particular antigen to induce an immune response. One of ordinary skill in the art would readily construct a CAR with particular specificity and function. In several embodiments, to engineer CAR T cells from an individual's own T cells, the T cells are harvested from the individual and desirable CAR construct is induced into the T cell, which can subsequently be returned to the individual as part of a treatment. In several embodiments, further treatments may be performed on the individual based on the individual's particular cancer or neoplasm, such as (for example) surgery, immunotherapy, chemotherapy, radiation therapy, targeted therapy, hormone therapy, stem cell therapies, and blood transfusions.

Throughout the description, the terms neoplasm, tumor, or cancer (or neoplastic cell and cancer cell) are utilized interchangeably. A neoplasm, tumor, or cancer, as understood in the field, is a new and abnormal growth of tissue, and thus includes benign growths (e.g., benign tumors) and cancerous growths. Similarly, a cancer is an abnormal growth of cells with the potential to metastasize and to spread to other areas of the body. Accordingly, the various embodiments described herein can be applied to neoplasms and cancers, unless specified to be exclusive to one or the other.

The engineered oncolytic viruses and engineered T cells described herein can be administered to an individual in accordance with any appropriate treatment regime. In some embodiments, an oncolytic virus is administered to reach the site of neoplastic cell growth. In some embodiments, an oncolytic virus is administered via an intratumoral delivery. In various embodiments, an oncolytic virus is administered via an enteral or a parenteral delivery, such as an intravenous, an intramuscular, or a subdermal delivery. The oncolytic virus may be dispersed in a pharmaceutically acceptable formulation for injection. In various embodiments, an individual is administered an order of 10⁵, 10⁶, 10⁷, 10⁸, 10⁹, 10¹⁰, 10¹¹, 10¹², or 10¹³ pfu of virus. In various embodiments, an individual is administered an oncolytic virus multiple times, including 1, 2, 3, 4, 5, 6, or more times. In some embodiments, the engineered CAR T cells are administered via intravenous, intra-arterial, or intralymphatic delivery.

As used herein, the term “pharmaceutical composition” relates to a composition for administration to an individual. In some embodiments, a pharmaceutical composition comprises the oncolytic virus described herein for enteral or parenteral administration, or for direct injection into a neoplasm. In some embodiments, a pharmaceutical composition comprising the engineered oncolytic virus or engineered CAR T cells described herein is administered to the individual via infusion or injection.

In some embodiments, oncolytic virus and/or CAR T cells are administered in a therapeutically effective amount as part of a course of treatment. As used in this context, to “treat” means to ameliorate at least one symptom of the disorder to be treated or to provide a beneficial physiological effect. For example, one such amelioration of a symptom could be reduction of tumor size.

A therapeutically effective amount can be an amount sufficient to prevent reduce, ameliorate or eliminate the symptoms of cancer. In some embodiments, a therapeutically effective amount is an amount sufficient to reduce the growth of neoplasm and/or metastasis of a cancer.

In accordance with various embodiments, numerous types of neoplasms or cancer can be treated by the compositions and methods disclosed herein. Neoplasms that can be treated include, but not limited to, acute lymphoblastic leukemia, acute B-lymphocytic leukemia, chronic non-lymphocytic leukemia, non-Hodgkin's lymphoma, anal cancer, astrocytomas, basal cell carcinoma, bile duct cancer, bladder cancer, breast cancer, breast adenocarcinoma (BRCA), cervical cancer, chronic myeloproliferative neoplasms, colorectal cancer, endometrial cancer, ependymoma, esophageal cancer, diffuse large B-cell lymphoma (DLBCL), esthesioneuroblastoma, Ewing sarcoma, fallopian tube cancer, gallbladder cancer, gastric cancer, gastrointestinal carcinoid tumor, hepatocellular cancer, hypopharyngeal cancer, Kaposi sarcoma, kidney cancer, Langerhans cell histiocytosis, laryngeal cancer, liver cancer, lung cancer, melanoma, Merkel cell cancer, mesothelioma, mouth cancer, neuroblastoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, testicular cancer, thymoma, thyroid cancer, uterine cancer, vaginal cancer, and vascular tumors.

The terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “a virus” may include a plurality of viruses, including mixtures thereof.

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### Example 1

Construction of a Recombinant VSV Attenuated Strain Carrying Full Length CD19 or CD19 with Truncation

### Construction of M Matrix Protein Gene Mutation Plasmid

With reference to the wild-type VSV strain MN164438.1, the attenuated strain with M gene mutation was screened by random mutation method. In one embodiment, the amino acid substitution(s) of M protein comprises one or more of N32S, N49D, M51R, H54Y, V221F, V225I, and S226R. i.e., the amino acid substitution of the M protein comprises one or more of the following: asparagine in position 32 was mutated to serine (N32S); the asparagine in position 49 was mutated to aspartic acid (N49D); the methionine in position 51 was mutated to arginine (M51R); the histidine in position 54 was mutated to tyrosine (H54Y); the valine in position 221 was mutated to phenylalanine (V221F); the valine at position 225 was mutated to isoleucine (V225I); the serine at position 226 was mutated to arginine (S226R). The method is briefly described as follows: First, point mutations to the M gene were performed with the Thermo Mutation Generation System Kit to obtain a pVSV plasmid library of M gene mutations. After virus rescue, the M mutation VSV virus strain was obtained, and in vitro and in vivo screening experiments were done to obtain attenuated strains with high oncolytic and weak pathogenicity. The amino acid sequence of the M matrix protein of the wild-type VSV virus strain MN164438.1 is shown in SEQ ID NO:1. The amino acid sequence of the mutant M matrix protein of the mutant attenuated strain is shown in SEQ ID No: 2.

### Construction of G Protein Gene Mutation Plasmid

A G protein gene mutation plasmid was constructed on the basis of the M mutation plasmid described above. The wild-type VSV strain MN164438.1 was used as a template to introduce mutation sites. The specific mutation comprises one or more of the following: the valine (V) at position 53 was mutated to isoleucine (I); the alanine (A) at position 141 was mutated to valine (V); aspartic acid (D) at position 172 was mutated to tyrosine (Y); lysine (K) at position 217 was mutated to glutamic acid (E); aspartic acid (D) at position 232 was mutated to glycine (G); valine (V) at position 331 was mutated to alanine (A); valine (V) at position 371 was mutated to glutamic acid (E); glycine (G) at position 436 was mutated to aspartic acid (D); threonine (T) at 438 was mutated to serine (S); phenylalanine (F) at 453 was mutated to leucine (L); threonine (T) at position 471 was mutated to isoleucine (I); tyrosine (Y) at position 487 was mutated to isoleucine (I). Molecular cloning methods were used to connect the G protein mutation gene to the skeleton vector (the M protein mutation plasmid vector mentioned above) from which the original G protein gene was removed, thereby constructing the G protein and M protein mutations plasmid vector. The amino acid sequence of the G protein of the wild-type VSV virus strain MN164438.1 is shown in SEQ ID NO:3. The amino acid sequence of a mutated G protein of VSV is shown in SEQ ID NO:4.

### Virus Rescue

According to MOI=5, BHK-21 cells were inoculated with poxvirus vTF7-3 expressing T7 RNA polymerase. After infection for 1 hour, the BHK-21 cells were rinsed once with DPBS buffer. Then prepare the plasmid transfection master mix, which specifically includes: pBS-N, pBS-P, pBS-L, and the mutant plasmid prepared above. Among them, pBS-N, pBS-P, and pBS-L refer to the expression plasmids in which the VSV N, VSV P, and VSV L protein genes are cloned, respectively, and express the N, P, and L proteins required for virus rescue. Plasmid transfection was carried out according to the method described in the lipofectamine 2000 instruction manual. After 4 hours, it was replaced with fresh DMEM complete medium containing 10% fetal bovine serum. After 48 hours, the supernatant was aspirated, and the pox virus was filtered with a 0.22 μm filter. The filtrate was added to fresh BHK-21 cells; the cytopathic condition was observed every day, and the supernatant was collected when the cells showed pathological changes. After confirmation by RT-PCR, the virus was purified by virus plaque experiment. The attenuated strain was obtained.

### Construction of Recombinant VSV Vector (VSV-CD19) Carrying Full Length CD19 or Truncated CD19

Artificially synthesized CD19 full-length sequence with restriction enzyme sites Xho I and Mlu I (amino acid sequence SEQ ID NO:5) or truncated CD19 sequence (amino acid sequence SEQ ID NO:6 or 7) were cloned into the non-coding region between the G protein and the L protein of the M matrix protein attenuated mutant strain prepared in Example 1 to obtain a recombinant VSV plasmid carrying full length CD19 or truncated CD19 polypeptide. The map of the VSV-CD19 vector is shown in FIG. 1. The VSV-CD19 vector was rescued as described above.

### Construction of Recombinant VSV Vector (VSV-IL12-CD19) Carrying IL-12 and Full Length CD19 or Truncated CD19

Artificially synthesized IL12 sequence with restriction enzyme sites Xho I and Mlu I (amino acid sequence SEQ ID NO:8) and full length CD19 sequence (SEQ ID NO:5) or truncated CD19 sequence (amino acid sequence SEQ ID NO:6 or 7) were cloned into the non-coding region between the G protein and the L protein of the M matrix protein attenuated mutant strain prepared in Example 1 to obtain a recombinant VSV plasmid carrying full length CD19 or truncated CD19 polypeptide. The map of the VSV-IL12-CD19 vector is shown in FIG. 2. The VSV-CD19 vector was rescued as described above.

### Example 2

Construction and Preparation of CD19 CAR Lentiviral Vector

A CD19 CAR sequence composed of CD8 signal peptide, CD19 antibody (FMC63) single-chain variable region, hinge region, transmembrane region, 4-1BB intracellular region and CD3 ζ chain was artificially synthesized in Suzhou Jinweizhi Biotechnology Co., Ltd. (nucleotide sequence SEQ ID NO:9, amino acid sequence SEQ ID NO:10) and was cloned to the lentivirus PCDH-EF1α-MCS-T2A-copGFP through cloneing sites Nhe I and Sal I.

The CD19 CAR lentiviral vector and its auxiliary vectors psPAX2 and PMD2.G plasmids were extracted and quantified using Tiangen endotoxin-free plasmid mass extraction kit.

### Example 3

CD19 CAR Lentivirus Preparation

Transfection of 293T cells: Day 0 Plating: 0.25% trypsin digestion of 293FT cells, plated in a 10 cm petri dish containing 10 ml DMEM (containing 10% FBS), cultured in a 37° C., 5% CO2 constant temperature incubator, cell confluence reached 90-95% after 24 hours.

Two hours before transfection, the 293FT cell culture medium was changed. Mix the CD19 CAR lentiviral vector and auxiliary vector with PEI, pipette evenly, and let stand at room temperature for 15 min to obtain a DNA/PEI mixture. Add the prepared DNA/PEI mixture dropwise to 293FT cells, continue to incubate for 4-6 hours, and change the medium.

The cell supernatant was collected 24 hours and 48 hours after transfection, filtered by a 0.45 μM filter, and ultracentrifuged at 25,000 rpm, 4 degrees, for 2 hours. Finally, dissolve the virus pellet with PBS.

### Example 4

CD19 CAR-T Cell Preparation

Use Ficoll lymphocyte separator to separate peripheral blood mononuclear cells (PBMC). Activate T cells for 24 hours with CD3 and CD28 antibodies. After T cell activation, the T cells were infected with the prepared lentivirus at a ratio of MOI=5. Seventy two hours after infection, use the Shanghai Nearshore CAR19 Detection Kit to perform flow immunofluorescence staining, analyze the infection efficiency of CD19 CAR, and confirm expression of CD19 CAR on T cells. After confirming the expression, continue to culture in X-VIVO15 serum-free medium containing IL-2 (500 U/ml) for 10 to 14 days to complete the preparation of targeted chimeric antigen receptor T cells.

### Example 5

Expressing CD19 or Truncated CD19 to Provide CD19 CAR-T Target
(1) Collect K562 cells (negative for CD19 expression), and adjust the cell concentration to 2×10⁷/ml after washing with PBS;
(2) Add 5 mM eFlouor670, the final concentration is 504, 37 degrees water bath to avoid light for 10 min;
(3) Add 6 times the volume of 1640 complete medium, incubate on ice for 5 min, stop labeling;
(4) Wash with PBS, 10 ml, 3 times;
(5) Resuspend the labeled K562 cells with X-VIVO15 (5% human AB serum) and adjust the cell concentration to 1×10⁶/ml.
(6) Take 1 ml of labeled K562 cells, namely 1×10⁶ cells were incubated at different MOIs (e.g. MOI 0, 1, 2, 4) with recombinant VSV virus expressing CD19 in X-VIVO15 (5% human AB serum) in a 24-well plate for 8 hours, and then washed with PBS to change the medium. Half of the cells were stained with 7-AAD to assess cell viability; at the same time, the other half cell samples were stained with anti-CD19 to assess CD19 expression by flow cytometry. As shown in FIG. 4, as the amount of virus increases, the positive rate of 7-AAD in K562 cells increases, indicating a decrease in cell viability. As shown in FIG. 5, as the amount of recombinant VSV virus increases, the rate of K562 cells expressing CD19 increases. When the MOI is 1, the positive rate is as high as 94.3%.

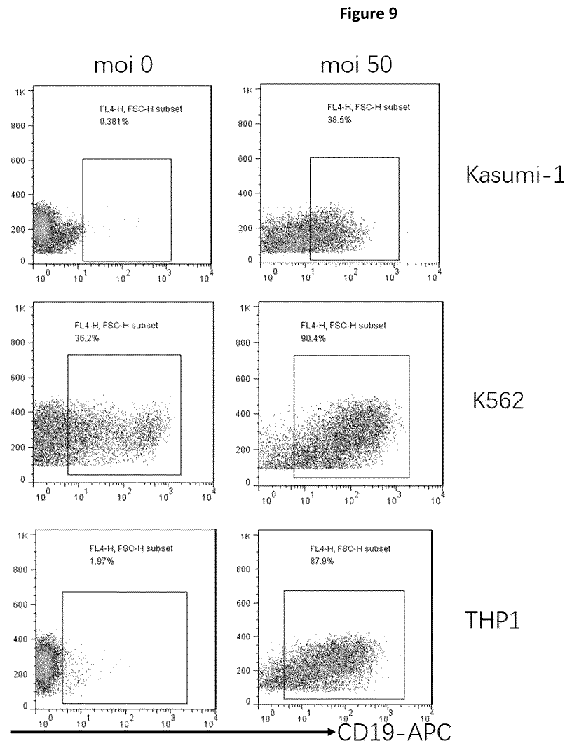

FIG. 9 shows the results of another experiment of inducing CD19 expression on CD19-negative tumor cell lines by recombinant VSV virus encoding CD19. Kasumi-1 cells are acute myeloid leukemia cells; K562 cells are erythroleukemia cells; and THP1 cells are myelomonocytic leukemia cells.

### Example 6

In Vitro Killing of CD19 CAR-T Cells Against K562 Cells Infected with Recombinant VSV Virus Expressing Full Length or Truncated CD19
(1) The K562-CD19 stable strain and CAR T cells were incubated at different target ratios of 1.25:1, 2.5:1, and 5:1. The cell groups were: CD19 CAR-T cells only, CD19 CAR-T cells and target cells loaded with CD19 full-length recombinant VSV (CD19 CAR-T cells+CD19-VSV), CD19 CAR-T cells and target cells loaded with recombinant VSV expressing truncated CD19 (CD19 CAR-T cells+TrunCD19-VSV), Recombinant VSV virus only (CD19-VSV), recombinant VSV virus with truncated CD19 only (TrunCD19-VSV), and untransduced T cells. Simply add 100111 effector cells per well to target cells. Incubate the cells in triplicates for 16 to 24 hours (the specific time is determined by microscopic observation of the tumor-killing effect, and the specific time of the oncolytic virus group is consistent with the experimental group). Include wells with target cell only and wells with effector cells only as controls for spontaneous death.
(2) After the tumor-killing incubation is completed, follow the experimental steps of Promega's CytoTox-GloTM Cytotoxicity kit, add 50 ul cytotoxixity assay reagent to each well, incubate at room temperature for 15 minutes, and read in a microplate reader.

After the reading is completed, add 50 ul Lysis reagent to each well, incubate at room temperature for 15 min, and read in a microplate reader.
(3) Calculate the killing efficiency according to the following formula:

Cytotoxicity %=RLU_{Experimental}−RLU_{Effector spontaneous}−RLU_{Target spontaneous}/RLU_{Target maximum}−RLU_{Target spontaneous}

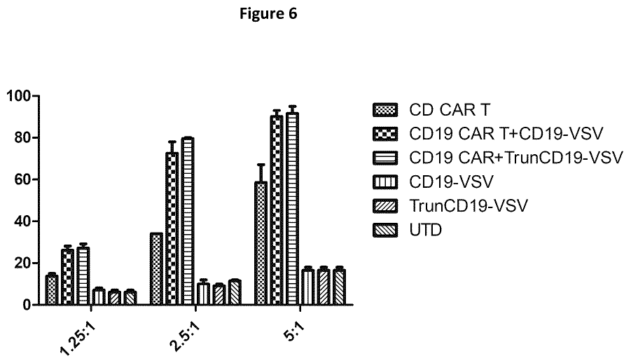

FIG. 6 shows in vitro killing of CD19 CART cells against target cells expressing full-length or truncated CD19. The combined application of CD19 CAR T and VSV expressing CD19 significantly increased the killing efficiency compared with CD19 CAR-T cells alone.

### Example 7

Therapeutic Effects in B Cell Lymphoma Model in Mice

(1) Using CRISPR Gene Knockout Technology to Knock Out CD19 Expression in Human B-Cell Lymphoma.

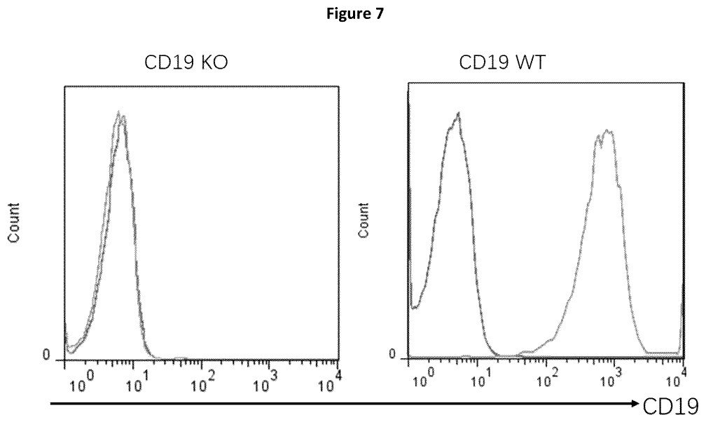

Forward primer for CD19 sgRNA: 5′CACCGATGAAAAGCCAGATGGCCAG 3′ (SEQ ID NO:11). Reverse primer for CD19 sgRNA: 5′AAACCTGGCCATCTGGCTTTTCATC 3′ (SEQ ID NO:12). Constructed into the Px330 vector, and transferred to the mouse B-cell lymphoma cell line Raji. Seventy two hours later, transfer the electroporated cells to a 96-well plate using the limiting dilution method. When the cell clones were grown, used flow immunofluorescence staining to detect the expression of CD19 on the cell surface, and selected clones with negative CD19 expression (see FIG. 7). Expand the culture and leave it for the following experiment.

(2) Mouse experiment

Select 6-8 Weeks Old B-NSG Female Mice, and Subcutaneously Inoculate the Mice with Raji negative for CD19 expression, 2×10⁶/mouse. Begin treatment when the size of the transplanted tumor grew to 10 mm³. Recombinant VSV virus was injected intratumorally, 10⁸ pfu/mouse, and CD19 CAR T cells were injected into the tail vein after 24 hours, 1×10⁷/mouse, see Table 1 for specific design.

TABLE 1
Treatment Groups

| | CAR | Recombinant |
|---|---|---|
| Groups | T cells | VSV virus (Pfu) |
| Simple CD19 CAR T injection | 1 × 10⁷ | 10⁸ |
| group | | |
| CD19 CAR T + CD19 full | 1 × 10⁷ | 10⁸ |
| length recombinant VSV virus | | |
| CD19 CAR T + truncated CD19 | 1 × 10⁷ | 10⁸ |
| recombinant VSV virus | | |
| Untransduced T cells (UTD) | 1 × 10⁷ | 10⁸ |
| Pure CD19 full-length | 1 × 10⁷ | 10⁸ |
| recombinant VSV virus | | |
| Simple truncation of CD19 full- | 1 × 10⁷ | 10⁸ |
| length recombinant VSV virus | | |

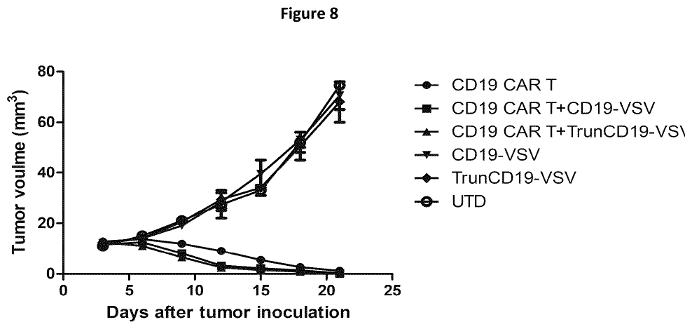

Measure tumor volume with a vernier caliper every two days. As shown in FIG. 8, tumor volume was significantly reduced in the combined treatment groups of CD19 CAR T cells and recombinant VSV virus as compared with groups treated with CD19 CAR T cell alone, or recombinant VSV virus alone.

What is claimed is:

1. A method of treatment of an individual having a cancer, comprising the steps of:
administering to the individual an oncolytic virus comprising nucleotide sequences encoding an antigen, or an antigen and IL-12; and
administering to the individual genetically engineered T cells expressing a chimeric antigen receptor (CAR) that recognizes the antigen.

2. The method of claim 1, wherein the antigen is CD19.

3. The method of claim 2, wherein the CD19 antigen comprises the amino acid sequence of SEQ ID NO: 5, 6 or 7.

4. The method of claim 1, wherein the CAR comprises the amino acid sequence of SEQ ID NO: 10.

5. The method of claim 1, wherein the T cells are obtained from an individual autologously or allogeneically.

6. The method of claim 1, wherein the oncolytic virus comprises M protein comprising one or more amino acid mutations of N32S, N49D, M51R, H54Y V221F, V225I and S226R as compared with SEQ ID NO. 1.

7. The method of claim 6, wherein the M protein comprises the amino acid sequence of SEQ ID NO:2.

8. The method of claim 1, wherein the oncolytic virus comprises G protein comprising one or more amino acid mutations V53I, A141V, D172Y, K217E, D232G, V331A, V371E, G436D, T438S, F453L, T471I, and Y487I.

9. The method of claim 8, wherein the G protein comprises the amino acid sequence of SEQ ID NO: 4.

10. The method of claim 1, wherein the oncolytic virus is vesicular stomatitis virus (VSV).

11. The method of claim 1, wherein the oncolytic virus is administered parenterally or intratumorally.

12. The method of claim 11, wherein the oncolytic virus is administered intravenously, subcutaneously or intramuscularly

13. The method of claim 1, wherein the engineered T cells are administered via intravenous, intra-arterial, or intralymphatic delivery.

14. The method of claim 1, wherein the individual is further treated with surgery, immunotherapy, chemotherapy, radiation therapy, targeted therapy, hormone therapy, stem cell therapies, or blood transfusions.

15. The method of claim 1, wherein the cancer is acute lymphoblastic leukemia, acute B-lymphocytic leukemia, chronic non-lymphocytic leukemia, or non-Hodgkin's lymphoma.


SEQUENCE LISTING (12 sequences)

SEQ ID NO: 1 - protein, 229 aa, Vesiculovirus indiana
MSSLKKILGLKGKGKKSKKLGIAPPPYEEDTNMEYAPSAPIDKSYFGVNEMDTHDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSVSHFK

SEQ ID NO: 2 - protein, 229 aa, synthetic construct
MSSLKKILGLKGKGKKSKKLGIAPPPYEEDTNMEYAPSAPIDKSYFGVNERDTHDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSVSHFK

SEQ ID NO: 3 - protein, 511 aa, Vesiculovirus indiana
MKCLLYLAFLFIGVNCKFTIVFPHNQKGNWKNVPSNYHYCPSSSDLNWHNDLVGTALQVKMPKSHKAIQADGWMCHASKWVTTCDFRWYGPKYITHSIRSFTPSVEQCKESIEQTKQGTWLNPGFPPQSCGYATVTDAEAAIVQVTPHHVLVDEYTGEWVDSQFINGKCSNDICPTVHNSTTWHSDYKVKGLCDSNLISMDITFFSEDGELSSLGKKGTGFRSNYFAYETGDKACKMQYCKHWGVRLPSGVWFEMADKDLFAAARFPECPEGSSISAPSQTSVDVSLIQDVERILDYSLCQETWSKIRAGLPISPVDLSYLAPKNPGTGPVFTIINGTLKYFETRYIRVDIAAPILSRMVGMISGTTTERVLWDDWAPYEDVEIGPNGVLRTSSGYKFPLYMIGHGMLDSDLHLSSKAQVFEHPHIQDAASQLPDGETLFFGDTGLSKNPIEFVEGWFSSWKSSIASFFFTIGLIIGLFLVLRVGIYLCIKLKHTKKRQIYTDIEMNRLGK

SEQ ID NO: 4 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 5 - protein, 557 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 6 - protein, 313 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 7 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 8 - protein, 603 aa, unidentified
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 10 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 25 bp, synthetic construct
  source 1..25: mol_type = other DNA
caccgatgaaaagccagatggccag

SEQ ID NO: 12 - DNA, 25 bp, synthetic construct
  source 1..25: mol_type = other DNA
aaacctggccatctggcttttcatc